(19) United States
(12) Patent Application Publication
(10) Pub. No.: US 2008/0147518 A1
(43) Pub. Date: Jun. 19, 2008

(54) Method and apparatus for pharmacy inventory management and trend detection

(75) Inventors: Sultan Haider, Erlangen (DE); Georg Heidenreich, Erlangen (DE)

Correspondence Address: SCHIFF HARDIN, LLP;PATENT DEPARTMENT, 6600 SEARS TOWER, CHICAGO, IL, 60606-6473, US

(73) Assignee: Siemens Aktiengesellschaft

(21) Appl. No.: 11/583,612

(22) Filed: Oct. 18, 2006

(51) Int. Cl. G06Q 10/00

(52) U.S. Cl. 705/28

(57) ABSTRACT

A method and system for inventory management of pharmacies and for detecting trends includes a pharmacy search and order service that receives information on prescriptions and communicates the prescription information to the pharmacies. The pharmacy search and order service also receives patient information, such as patient demographic data. The prescription information and patient information received by the pharmacy search and order service is transferred to a self-learning service where a rule database analyzes the information to detect trends in usage of medical items. The pharmacy search and order service determines which pharmacies have requested medical items in inventory and provides user output information indicating availability of the medical items at the pharmacies or indicates a nearest pharmacy having the items. An option provides for confirmation from the pharmacy of the item in inventory.

### BACKGROUND OF THE INVENTION

1. Field of the Invention

The present invention relates generally to a method and apparatus for managing the inventory of one or more pharmacies.

2. Description of the Related Art

A pharmacy may have little or no problem maintaining a stock of commonly used medicine and medical items, as these are frequently requested and the pharmacy could expect to keep them on hand. However, there is a problem when unusual medications or medical items or unusual quantities of medications are required by the patient, as these may not be on hand at the pharmacy.

Further, acute patients that need medications may be located in inaccessible areas, or at least areas in which access is more difficult. If the pharmacies know about the demand of certain medicaments in a certain geographic region, delays in obtaining and providing the medications and medical supplies could be avoided.

### SUMMARY OF THE INVENTION

The present invention provides a method and system for managing inventory quantities in pharmacies and other medical item dispensing entities, and in a preferred embodiment for detecting trends for the development of inventories.

The method and system manages the inventories in the pharmacies or other entities that dispense medical items and detects trends in the use of medicines and medical devices such as determining what is the relevance of the medicament being requested and for which kind of patient in a region. This is accomplished by making use of a pharmacy search and order service for receiving patient and prescription information. The system may include a self learning service for determining information contained in the patient and prescription information. By applying the present method, the pharmacy gets the knowledge about the usage of medicament during a patient care process. Hence the pharmacy service could maintain the inventory of needed medications, medicaments and medical items for patients with certain symptoms, thus decreasing delays.

### BRIEF DESCRIPTION OF THE DRAWINGS

FIG. 1 is a schematic representation of the method according to the principles of the present invention.

### DETAILED DESCRIPTION OF THE PREFERRED EMBODIMENTS

The present invention provides a method and system for gathering patient and prescription information and providing user output to identify one or more pharmacies having the prescribed medical item. The user output may include an identification of a nearest or a set of nearer pharmacies having the prescribed item. The output may include an identification of at least one pharmacy having the prescribed item. The output may include a confirmation that one or more pharmacies has the prescribed item in inventory.

The present invention also provides a method and system for monitoring demand for medicines and medical items at pharmacies and other medical item dispensing entities for utilizing the knowledge about the usage of the medical items to identify trends so that the pharmacy or other entity may react accordingly. The pharmacy or other entity may utilize knowledge of the trend to anticipate higher or lower demand for particular medical items and medicines. Preferably, the pharmacy can change its inventory of the medicines, medicaments and medical items to meet the anticipated demand as identified by the trend.

The present method uses the records of orders, or prescriptions, submitted to pharmacies to determine trends in usage of the medical items being purchased. These trends may indicate the needs of a particular patient or group of patients in an area near a pharmacy, may indicate an increase or decrease in an ailment or disease, or may indicate a preference for a particular treatment or medicament in one region compared to another. Other reasons for such trends may be found as well. The trends assist the pharmacies in managing their inventories. For example, a projected increased need for a particular medical item can be used by the pharmacy to ensure that more of that medical item is on hand. A decreased forecasted need enables the pharmacy to permit its stocks or inventory of that medical item to decline or even ship some of the medicine from the pharmacy. Regional needs are tracked and movement of inventories to pharmacies in a region or between pharmacies may be implemented to meet a growing need for particular items in that region or location. More than regional need, needs of patients near particular pharmacies may be determined so that a patient or group of patients ordering a particular medical item or medicine or class of medical item or medicine do not exhaust that pharmacies stock or inventory.

The trend analysis may take into account the ailment or malady that the prescribed medical items are meant to address. Other medicines, medicaments or medical items that are used for this particular ailment or malady may be required by the patient or by other patients, so that the present invention determines that the treatment of a particular malady follows a course of medicines, for example, and an upswing in orders for a medicament used in the early treatment stages of a disease may be identified and associated with the need to stock items associated with later treatment stages of the malady.

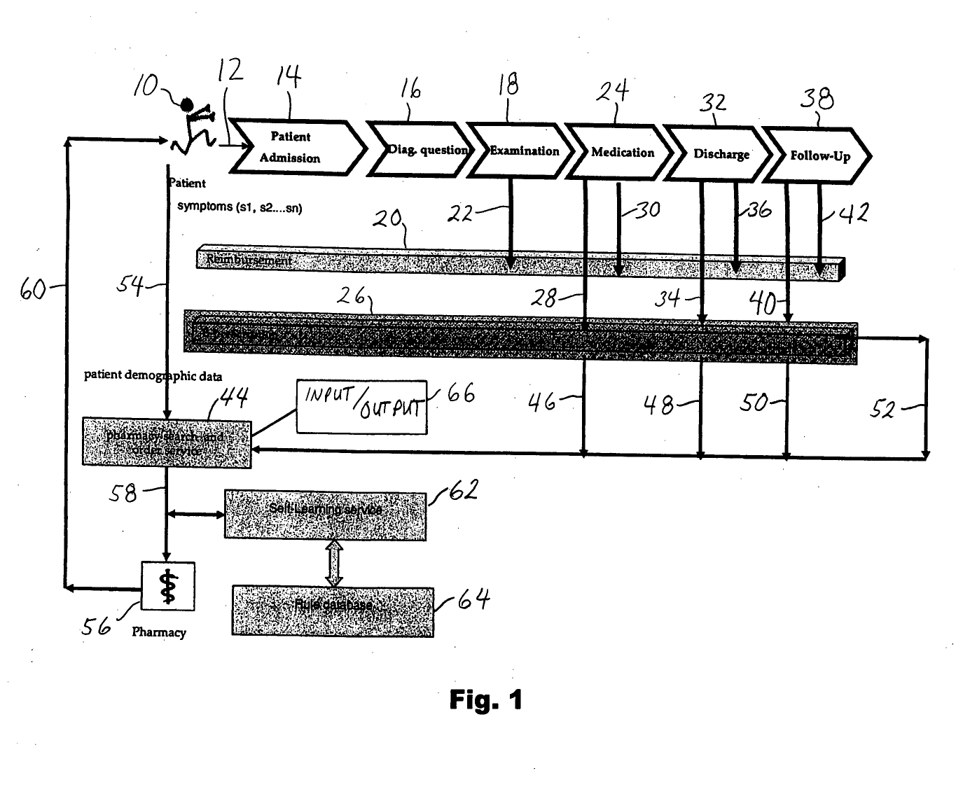

As shown in FIG. 1, a patient 10 enters a care path 12. The care path 12 includes the steps typically taken in patient care, although other steps are of course possible. In the illustrated care path 12, the patient 12 who may be afflicted with symptoms visits a health care facility and is admitted to the facility in a patient admission step 14. This is followed by diagnostic questions 16 and then an examination 18. The examination step 18 provides information on the processes performed during the examination 18 to a reimbursement channel 20 at 22. In the typical care path 12, the examination 18 is followed by a medication step 24. The medication step 24 of the illustrated embodiment provides an electronic prescription, or e-prescription, to an e-prescription channel 26, as indicated at 28. Also during the medication step 24, input is provided to the reimbursement channel 20, as indicated at 30. The patient 10 is hopefully discharged from the medical facility at discharge step 32, which may result in further input to the e-prescription channel 26, as shown at 34, and may result in input to the reimbursement channel 20, as shown at 36. The medical care professionals at the medical care facility may have the patient 10 follow up on the outcome of the treatment at a follow-up step 38. The follow-up step 38 can result in further e-prescriptions 26 being prepared, as shown at 40 and will generally result in input to the reimbursement channel 20 as shown at 42.

The reimbursement channel 20 includes billing for the services and supplies, submitting information to insurance companies, receiving payments, and other reimbursement activities, which are not shown in detail here. The e-prescription channel 26, on the other hand, forwards the information it receives regarding electronic prescriptions to a pharmacy search and order service 44. The information on the electronic prescriptions may be provided to the search and order service 44 each time the e-prescription channel 26 receives an e-prescription, as shown at 46, 48 and 50, corresponding to the e-prescription information instances 28, 34 and 40. It is also possible that a single transfer from the e-prescription channel 26 to the search and order service 44, as shown at 52, be made at some time not coinciding with receipt of an e-prescription by the e-prescription channel 26.

The pharmacy search and order service 44 also receives patient demographic information, as indicated at 54, either directly from the patient 10 or from another source by permission of the patient. The pharmacy search and order service 44 in the present embodiment forwards the patient information and e-prescription information to a pharmacy 56 as shown at 58. The pharmacy 56 fills the prescription by providing the medicament or medical item to the patient 10, as indicated at 60.

The filling of the prescription as indicated at 60 may be by providing one or more medicines, medicaments, medical items or medical appliances to the patient or for the patient's use. The present invention is not limited to the item or type of item supplied by the pharmacy or to the patient, but may be anything from which a trend analysis can be made or anything that may be tracked for inventory management. The invention is also not limited by the form of the order or prescription, so that the prescription may be an electronic prescription or e-prescription, a written or paper prescription, or some other form of order or prescription or designation of items for the patient. The patient may be a medical patient, dental patient, psychiatric patient or other patient, or may be a person or entity in general and may even not be a patient at all.

The pharmacy 56 may be a local pharmacy or drug store, a mail order pharmacy, a medical supply house, a specialty medical equipment supply, or any type of entity that provides items to patients or that fills orders for patients or other individuals.

The pharmacy search and order service 44 of a preferred embodiment has knowledge of whether the pharmacy 56 has particular medical items in stock. This knowledge may be the result of the search and order service 44 maintaining a record of the pharmacy stock or inventory, the result of the pharmacy 56 transmitting stock data to the search and order service 44 at predetermined times, or the result of the search and order service 44 requesting information from the pharmacy 56 relating to its inventory, such as upon receipt of a prescription for that item or determination of a need for the item as indicated by a trend.

The pharmacy 56 as shown represents a plurality of pharmacies in an embodiment of the invention. By including a plurality of pharmacies, the present invention is able to determine which of the pharmacies is closest to the patient, or which pharmacy has the desired item in stock. The identification of closest pharmacies may instead identify a set of pharmacies that are closer to the patient, thereby giving the patient a choice of nearby pharmacies at which to obtain the item. By connecting to a plurality of pharmacies, a larger base of data can be collected for determining trends and a greater area can be polled for pharmacies having the desired items in stock. Preferably, the pharmacies are distributed over a region or may even be nationwide.

Similarly, the patient 10 represents a plurality of patients and preferably the health care facility represents a plurality of health care facilities. The larger the sample of patients, the better the trend analysis so that a large sampling of patient data is desirable.

Of particular significance to the present invention, the output of the pharmacy search and order service 44 is provided to a self-learning service 62. The self-learning service 62 is shown as connected in the communication channel 58 to the pharmacy 56, but it is also envisioned that the self-learning service is connected directly to the pharmacy search and order service 44 or to some other portion of the present system. The self-learning service may even be connected to receive the data from the e-prescription channel 26.

The self-learning service 62 communicates with a rule database 64 and provides feedback both to the self-learning service 62 as well as to the search and order service 44 and the pharmacy 56.

In further detail, the objectives of the present invention are accomplished by making use of a pharmacy search and order service 44 and the self learning service 62. By applying the present method, the pharmacy 56 gets the knowledge about the usage of medicament during the patient care process. Hence, the pharmacy service 56 could maintain the inventory of medications for treatment of patients 10 with certain symptoms, thus decreasing delays in obtaining the medications for these patients.

In a preferred embodiment, the self learning service 62 learns about the patient care paths 12 and updates the rule database 64 with the learned information. The rule database 64 consists of rules relating to the symptoms, medicaments or medical items, care paths, and time. For example, a rule in the rule database may provide that for a symptom, such as symptom s1, the patient needs a medicament m1, etc. during a care path c1 after time t1. Rules for each patient/care path/e-prescription are provided to the rules database.

As a result of the present invention, the patient is offered services for placing an order to the nearest pharmacy to get the quickest delivery of the medicaments. The patient is assured that even for obscure items the identified pharmacy or pharmacies will have the item in inventory, without requiring a special order or delay.

The proposed system comprises the pharmacy search and order service 44, the self learning service 62, and the rule database 64. Also included is an input and output means 66 for getting data into the system and for getting data out. The input and output 66 may be any of the known input and output means, such as a computer terminal or workstation, kiosk, telecommunication device, portable electronic device, or the like. The output means 66 is preferably a user output, such as a display screen or printer via which a user is informed of the result of the analysis. For instance, the user output 66 may be a display at the doctor's office reporting to the patient the location of a pharmacy having the required medicament in stock or in inventory. The input and/or output 66 may be at the health care facility, at the user's home, at a kiosk, or other location.

An authentication and authorization mechanism is provided for ensuring the identity of entities during communications. The authentication and authorization mechanisms are incorporated into the system in the communication channels as shown in the drawing and are preferably in place for all communications channels where communications are transferred between entities for which the identity of the entity is important, particularly for transmittal of critical or sensitive information.

The pharmacy search and order service 44 takes patient demographic data as an input as shown at 54 and looks for the nearest pharmacy 56 to the patient 10. This nearest pharmacy 56 is preferably the nearest pharmacy that has the required medicament or medical item in stock. The present system may instead output a set of nearer pharmacies when several pharmacies have the requested item in inventory. Additional information may also be provided to the user, such as directions to the pharmacy, the price of the requested item, special use directions or precautions, or other information.

During the care process 12, the pharmacy search and order service 44 gets inputs (or orders) via the e-prescription channel 26. It is also foreseen that the inputs to the pharmacy search and order service 44 may come directly from the medical care facility or from another source rather than being transmitted through a prescription service. For example, an input directly into the search and order service 44 is provided at 66.

There may be financial and non-financial incentives for using and offering such a service. For example, the pharmacy is made aware of trends in the area of the pharmacy and so can adjust inventory of goods to better meet those trends. Further, a pharmacy that has in stock a particular item needed by a particular patient is identified to the patient so that the patient is more likely to purchase the item from that pharmacy rather than ordering the item from a pharmacy that does not have it in current stock. The patient or medical care professional may be offered an incentive or discount for use of the present system. The cost savings of improved inventory management by the pharmacy may be passed along to the patient as lower prices, or some other incentive program.

The patient 10 may use the patient's electronic health card, insurance card, or other identification at an Internet portal or other place for getting assessing to the pharmacy search and order service 44 or to other portions of the present system. The medical professional may also access the service 44 or other portion of the system on behalf of the patient.

The pharmacies 56 get an additional advantage for planning the quantity of medicaments that should be present. For instance, when the rule database forecasts a likely need for a medicament, the inventory of the medicament are preferably increased.

For the self learning service 62, the system can make use of artificial neural networks, genetic algorithms, Bayesian rules, etc. Use of these known tools enables the self learning system to identify trends in use of medicines, but even more importantly to deduce likely symptoms or ailments that are being treated by the medicines or medical items and so predict usage for other medicines or medical items that might be prescribed for treatment of these symptoms or ailments. Increased call for medicines for treating age related diseases or conditions may enable the local pharmacy to stock up on other items for use by an aging population. Similarly, an increased call for medicines relating to pregnancy, childbirth and treatment of young children may enable a pharmacy to stock up on medications more commonly prescribed for small children. Outbreaks of particular diseases, such as a flu outbreak, could be recognized and flu treatments and medicines inventory increased for the pharmacies in the afflicted areas.

The self learning service helps in detecting trends for the usage of certain medicaments in certain care paths. As trends in medicine use, trends in disease occurrence, trends in population aging or health, trends in workplace or home injuries or ailments, or other trends are recognized, the inventory of the local pharmacies are changed to accommodate the changes.

The detected clinical trend could be used for improving the clinical, financial and operational performance of the treatment process. For example, if the trend predicts an outbreak of a disease or a trend to an aging or younger population, the information may be used not only by the pharmacies but also by the medical facilities to better deal with the projected issues.

In a first version of the present invention, for example, the physician or other health care worker places an order for prescribed items/medicine on behalf of the patient. As a precondition, the patient is in the doctors' office or has enabled and authorized the doctor to access records and to prescribe and order on behalf of the patient. The electronic prescription for that patient is already stored on the local physician's computer system or on a central server (e.g. part of a health telematics infrastructure) granting access to that physician.

An option in the first version based on nearby pharmacies is to use location information about the general practitioner's office location or the patients residence, and to determine a suitable set of nearby pharmacies that may be offered to the patient in an overview screen on a display in the general practioner's office so that the patient and/or the doctor may use this information to choose the pharmacy.

Another option in the first version based on availability of the medicine is to require an immediate reply from the pharmacy (when building up such an overview screen, before placing the order), to determine availability of the requested medicament or medical item and optionally the terms of payments/delivery of the item/medicine. This is provided in one example by soliciting information from a plurality of pharmacies as to which pharmacies have the requested medical item in stock. The pharmacies that have the item may provide a reply to the request. The inventory checking and reply according to this option is provided by a computerized inventory management system that is poled by the present system, such as by the search and order service. This information can be shown on the display of the general practitioner's office and is used to choose the pharmacy.

In one scenario in the first version, use is made of the already computerized electronic prescription data, and the patient is assisted by the doctor in choosing a pharmacy that accepts the order and is able to deliver the prescribed items/medicine. The patient orders electronically at the chosen pharmacy.

A further option based on confirmation is to use an immediate reply from the pharmacy (after placing the order) as the basis for determining the pharmacy to get the prescription order. A confirmation about the terms of payment and delivery is presented on the display at the general practitioner's office.

In an second version, a patient places an order for prescribed items/medicine using the hardware in or near the general practitioner's office. As a precondition, an electronic prescription for that patient is already stored on the local physician's computer system or on a central server (e.g. part of a health telematics infrastructure) granting access to that patient. In the general practitioner's office or nearby there is a terminal (for example, an “eKiosk” or electronic kiosk) that is accessible to patients which allows patients to order the items/medicine as specified in the pending electronic prescriptions at a pharmacy.

An option in the second version based on nearby pharmacies is to use location information about the general practitioner's office location or the patients residence, and determine a suitable set of nearby pharmacies that may be shown on an overview screen on the eKiosk and that is used to order.

Another option in the second version based on availability requires an immediate reply from the pharmacy as to the availability of the requested item at the respective pharmacy. Pharmacies having the requested item and desiring the order send a response. Those that either do not have the item in stock or that for other reasons do not wish the order need not reply. The reply from the pharmacies is used for generating an overview screen, for example, before placing the order. The availability and terms of payment and delivery of the item or medicine can be shown, for example, on the eKiosk being used to choose the pharmacy.

In one scenario of the second version, already computerized electronic prescription data is used, and the patient uses a terminal (such as an “eKiosk”) in order to choose a pharmacy that will accept the order and which will deliver the prescribed items/medicine. The patients orders electronically at the chosen pharmacy.

In the second version, an option based on confirmation provides for requiring an immediate reply from the pharmacy after placing the order and a confirmation about the terms of payment and delivery is presented on the eKiosk.

In a third version, the patient places an order for prescribed items/medicine using the hardware at his home. As a precondition, an electronic prescription for that patient is already stored on a central server (e.g. part of a health telematics infrastructure) granting access to that patient. A requirement is that the patient have special hardware or have security functionality for standard hardware so that the user's terminal (which is preferably implemented using a PC or other computer and which for purposes of the present invention is referred to as an “eKiosk”) is accessible to the patient at the patient's home and allows that patient to electronically order items or medicine as designated in pending electronic prescriptions stored on the central server at a pharmacy.

An option in the third version for identifying nearby pharmacies uses location information about the eKiosk location or the patient's residence, to designate a suitable set of nearby pharmacies that may be offered in an overview screen on the eKiosk being used to place the order.

Another option in the third version based on availability requires an immediate reply from the pharmacy (when building up such an overview screen, and before placing the order). The reply from the pharmacy may indicate the availability and terms of payment and delivery of the item or medicine and this reply can be shown on the eKiosk being used to choose the pharmacy.

In a scenario using the third version, already computerized electronic prescription data is provided to the patient and the patient uses the patient's terminal (here functioning as and referred to as an “eKiosk”) in order to choose a pharmacy that will accept the order and which will deliver the prescribed items or medicine. The patients orders electronically at the chosen pharmacy.

An option in the third version requiring a confirmation provides for requiring an immediate reply from the pharmacy after placing the order, a confirmation about the terms of payments/delivery is presented on the eKiosk.

Thus, there has been shown and described a method and system for monitoring inventory prescriptions, such as e-prescriptions, identifying pharmacies base on location, availability or confirmation for selection by a patient or doctor, and determining trends for use in anticipating changes in inventory.

Although other modifications and changes may be suggested by those skilled in the art, it is the intention of the inventors to embody within the patent warranted hereon all changes and modifications as reasonably and properly come within the scope of their contribution to the art.

We claim:

1. A system for managing inventory of a pharmacy, comprising:
a pharmacy search and order service connected to receive information relating to prescriptions, said pharmacy search and order service having an output at which prescription information is transmitted to at least one pharmacy;
a self learning service connected to receive prescription information from said pharmacy search and order service;
a rule database connected in communication with said self learning service, said rule database storing rules relating to prescription orders; and
a user output for communicating to a patient information relating to the prescription.

2. A system as claimed in claim 1, wherein said pharmacy search and order service receives patient demographic data as input data, said system operating to determine a nearest pharmacy to a patient at which a prescribed item is in inventory, said user output providing information on the nearest pharmacy with the item to a user.

3. A system as claimed in claim 1, wherein said system has data of the at least one pharmacy relating to inventory of medical items and medicine at said pharmacy.

4. A system as claimed in claim 3, wherein said pharmacy search and order service obtains said data relating to inventory of the at least one pharmacy by requesting the data relating at least to one medical item or medicament.

5. A system as claimed in claim 3, wherein said at least one pharmacy is a plurality of pharmacies, said system operates to identify at least one selected pharmacy of said plurality of pharmacies having a medical item or medicament in stock.

6. A system as claimed in claim 1, wherein said system identifies a trend and provides information to the at least one pharmacy relating to said trend.

7. A method for managing inventory of a pharmacy, comprising the steps of:
obtaining patient information of a plurality of patients;
receiving prescription information for prescriptions of ones of the plurality of patients;
analyzing the patient information and prescription information for trends; and
outputting information on the trends at a user output.

8. A method for managing inventory of a pharmacy, comprising the steps of:
obtaining patient information of a plurality of patients;
receiving prescription information for prescriptions of ones of the plurality of patients;
receiving inventory information of a plurality of pharmacies;
determining at least one pharmacy having a prescribed item in inventory using the patient information and the prescription information and the inventory information; and
outputting information regarding the at least one pharmacy to a user.

9. A method as claimed in claim 8, further comprising the step of:
receiving a confirmation of the prescribed item in inventory from the at least one pharmacy.

10. A method as claimed in claim 8, wherein said information regarding the at least one pharmacy is information on a nearest pharmacy to patient.

11. A method as claimed in claim 8, wherein said information regarding the at least one pharmacy is information on at least one pharmacy having a predetermined item in inventory.
